(19) United States
(12) Patent Application Publication
(10) Pub. No.: US 2026/0294904 A1
(43) Pub. Date: Oct. 1, 2026

(54) TABLET COMPRISING 6-((5-METHYL-3-(6-METHYLPYRIDIN-3-YL)ISOXAZOL-4-YL)METHOXY)- N-(TETRAHYDRAPYRAN-4-YL)PYRIDAZINE-3-CARBOXAMIDE

(71) Applicant: Hoffmann-La Roche Inc., Little Falls, NJ (US)

(72) Inventors: Felix DITZINGER, Muttenz (CH); Reto MAURER, Basel (CH)

(73) Assignee: Hoffmann-La Roche Inc., Little Falls, NJ (US)

(21) Appl. No.: 19/477,725

(22) PCT Filed: Apr. 22, 2024
(86) PCT No.: PCT/EP2024/060856

§ 371 Date: Oct. 22, 2025

(30) Foreign Application Priority Data
Apr. 24, 2023 (EP) 23169355.7

(51) Int. Cl. A61K 31/501; A61K 9/20; A61K 9/28; A61K 47/02; A61K 47/14; A61K 47/26; A61K 47/38

(52) U.S. Cl. CPC A61K 31/501; A61K 9/2095; A61K 9/2813; A61K 9/2826; A61K 9/2853; A61K 9/2866; A61K 9/2893; A61K 47/02; A61K 47/14; A61K 47/26; A61K 47/38

(57) ABSTRACT

The present invention relates to a tablet comprising 6-((5-methyl-3-(6-methylpyridin-3-yl)isoxazol-4-yl)methoxy)-N-(tetrahydropyran-4-yl)pyridazine-3-carboxamide, to processes for its preparation, kits comprising it, and its use in medical treatment.

### FIELD OF THE INVENTION

The present invention relates to a tablet comprising 6-((5-methyl-3-(6-methylpyridin-3-yl)isoxazol-4-yl)methoxy)-N-(tetrahydropyran-4-yl)pyridazine-3-carboxamide, to processes for its preparation, kits comprising it, and its use in medical treatment.

### BACKGROUND OF THE INVENTION

6-((5-methyl-3-(6-methylpyridin-3-yl)isoxazol-4-yl)methoxy)-N-(tetrahydropyran-4-yl)pyridazine-3-carboxamide (I), also known as alogabat (WHO Drug Information, Vol. 35, No. 2, 2021, 366), is a small molecule, selective gamma-aminobutyric acid type A (GABAA) 5 subunit-containing receptor positive allosteric modulator (PAM).

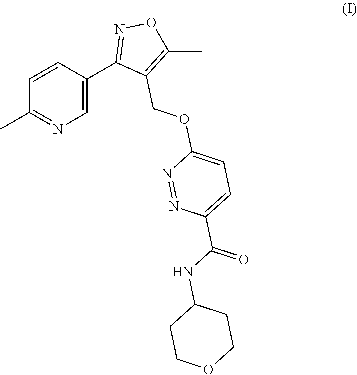 [SMILES: Cc1ccc(-c2noc(C)c2COc2ccc(C(=O)NC3CCOCC3)nn2)cn1]

Alogabat is currently in Phase II clinical trials to investigate its pharmacokinetics and safety in children and adolescents with Angelman Syndrome (AS), and to investigate its efficacy, safety, and tolerability in patients with autism spectrum disorder (ASD).

For Phase I clinical trials, alogabat was administered filled in hard capsules. However, hard capsules may not be suitable for administering the API to patients suffering from AS or ASD, especially pediatric AS or ASD patients, because they are more likely to suffer from anxiety disorders, and/or be more sensitive to tastes and textures than the average population, making it potentially difficult for them to swallow hard capsules. Therefore, an alternative to the Phase I capsule formulation was needed.

WO2018104419 discloses tablet formulations comprising alogabat. However, the tablet formulations disclosed in WO2018104419 suffer from the same drawbacks outlined above for the capsule formulation. In addition, it was found that using lactose as filler in tablet formulations, as described in WO2018104419, is incompatible with the physical properties of the API, especially at high drug loads (see Example 4). Furthermore, as part of the Phase I clinical studies, a taste assessment with five healthy volunteers was conducted, which revealed that the API has an unpleasant bitter taste. This may make swallowing tablets comprising alogabat even more challenging for patients having difficulties swallowing, such as pediatric AS and ASD patients.

In summary, there is an unmet need for new formulations of alogabat.

### SUMMARY OF THE INVENTION

As a solution to the problems outlined above in relation to the Phase I capsule formulation and the tablet formulations disclosed in WO2018104419, the present invention provides a tablet that can be dispersed in a liquid, such as water or apple juice, prior to administration. This greatly facilitates the administration of alogabat to ASD and Angelman syndrome patients, in particular pediatric ASD and Angelman syndrome patients. Dispersing the tablet in an acidic beverage, such as apple juice, also overcomes the problem of the unpleasant bitter taste of the API, since it was found that the API has a low solubility at acidic pH. In other words, when a tablet according to the invention is dispersed in an in an acidic beverage, such as apple juice, the API will not dissolve, preventing it from unfolding its unpleasant bitter taste. In order to promote the disintegration of the tablets in water and acidic beverages, respectively, a superdisintegrant was added to the formulation.

During the development of the tablet formulation of the invention, it was surprisingly found that, at high drug loads of >25%, lamination and capping occurred upon compressing the formulation blends into tablet kernels. The reason for this behavior might be found in the intrinsic mechanical properties of the API. Namely, it was found that the API exhibits an extreme plastic behavior even at low compaction pressures. Consequently, the tablets according to the invention have a drug load of ≤25%.

Furthermore, due to the plastic behaviour and low tensile strength value of the API, the API is not compatible with excipients that exhibit a low tensile strength- and a high compaction pressure value, such as lactose, especially in formulations with a high drug load. Excipients with low tensile strength- and a high compaction pressure value like lactose were therefore excluded when developing the tablet formulation according to the invention.

In summary, in a first aspect, the present invention provides a tablet comprising a kernel and optionally a coating, wherein said kernel comprises:
(i) the API 6-((5-methyl-3-(6-methylpyridin-3-yl)isoxazol-4-yl)methoxy)-N-(tetrahydropyran-4-yl)pyridazine-3-carboxamide (I), or a pharmaceutically acceptable salt thereof,

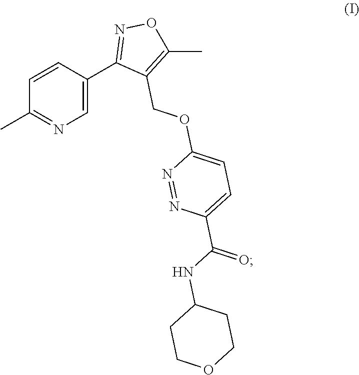 [SMILES: Cc1ccc(-c2noc(C)c2COc2ccc(C(=O)NC3CCOCC3)nn2)cn1]

(ii) a superdisintegrant;
(iii) a first filler;
(iv) a second filler;
(v) a glidant; and
(vi) a lubricant;
wherein the amount of API is ≤25%±1% relative to the total weight of the kernel,
preferably ≤20%±1% relative to the total weight of the kernel; and
wherein said kernel does not comprise lactose.

The tablets according to the invention can be manufactured on an industrial scale, have a low content standard deviation and show a fast disintegration.

### BRIEF DESCRIPTION OF THE FIGURES

FIG. 1 depicts a flow chart of a process for manufacturing a tablet according to the invention.

FIG. 2 depicts a flow chart of a process for manufacturing a tablet comprising a sweetener and a flavor according to the invention.

FIG. 3 depicts a flow chart of a process for manufacturing a tablet comprising a sweetener according to the invention.

FIG. 4 depicts dissolution profiles of tablets according to the invention with various dose strengths.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

As used herein, the term “dose strength” relates to the absolute amount of the compound of Formula I in its free base form contained in a tablet formulation according to the invention, expressed in milligrams (mg). Consequently, if the compound of formula I is used in the form of a pharmaceutically acceptable salt, the term “dose strength” relates to the respective free base equivalent.

As used herein, the term “filler” refers to a substance added to a pharmaceutical composition to increase the weight and/or size of the pharmaceutical composition. Pharmaceutically acceptable fillers are described in Remington's Pharmaceutical Sciences and listed in Handbook of Pharmaceutical Excipients, Sheskey et al., 2017. Non-limiting examples of fillers are starch (e.g., pregelatinized starch), cellulose (e.g., microcrystalline cellulose) and lactose (e.g., lactose monohydrate). Preferred, yet non-limiting examples of fillers are cellulose and lactose.

As used herein, the term “disintegrant” refers to a substance added to a pharmaceutical composition to help break apart (disintegrate), e.g., after administration, and release the active ingredient. Pharmaceutically acceptable disintegrants are described in Remington's Pharmaceutical Sciences and listed in Handbook of Pharmaceutical Excipients, Sheskey et al., 2017. Non-limiting examples of disintegrants are low substituted hydroxypropyl cellulose alginic acid. A preferred, yet non-limiting example of a disintegrant is alginic acid.

As used herein, the term “superdisintegrant” refers to highly effective disintegrants that, when used at low levels, provide rapid disintegration and dissolution of a tablet or capsule dosage form (see USP <1059> “Exipient performance”, as well as USP 41-NF 36 General Chapter <2040> “Superdisintegrants”). Preferred, yet non-limiting examples of superdisintegrants are croscamellose sodium and crospovidone. A particularly preferred example of a superdisintegrant is croscarmellose sodium.

As used herein, the term “lubricant” refers to a substance added to a pharmaceutical composition to help reduce the adherence of a granule of powder to equipment surfaces. Pharmaceutically acceptable lubricants are described in Remington's Pharmaceutical Sciences and listed in Handbook of Pharmaceutical Excipients, Sheskey et al., 2017. Non-limiting examples of lubricants are sodium stearyl fumarate and magnesium stearate. A preferred, yet non-limiting example of a lubricant is sodium stearyl fumarate.

As used herein, the term “glidant” refers to a substance added to a pharmaceutical composition to enhance product flow by reducing interparticulate friction. Pharmaceutically acceptable glidants are described in Remington's Pharmaceutical Sciences and listed in Handbook of Pharmaceutical Excipients, Sheskey et al., 2017. Non-limiting examples of glidants include silicon dioxide (colloidal), polyethylene glycol PEG 6000, fumed silicon dioxide Aerosil® 200, talc and the like. A preferred, yet non-limiting example of a glidant is colloidal silicon dioxide.

As used herein, the term “sweetener” refers to a substance providing sweet taste and has taste similar to a sugar to make the product more palatable. This includes natural and synthetic sugars, natural and artificial sweeteners, natural extracts, and any material that initiates a sweet sensation in a subject. Sweeteners illustratively include glucose, fructose, sucrose, xylitol, tagatose, sucralose, maltitol, isomaltulose, isomaltulose, lactitol, sorbitol, erythritol, trehalose, maltodextrin, polydextrose, and the like. Other sweeteners illustratively include glycerin, inulin, erythritol, acesulfame, and salts thereof, e.g., acesulfame potassium, alitame, aspartame, neotame, sodium cyclamate, saccharin and salts thereof, e.g., saccharin sodium or saccharin calcium, neohesperidin dihydrochalcone, stevioside, thaumatin, and the like. A particularly preferred sweetener is sucralose.

As used herein, the term “flavor” refers to a substance can be used to mask unpleasant tasting active ingredients and improve the acceptance that the patient will complete a course of medication. Flavorings may be natural (e.g. fruit extract) or artificial.

As used herein, the term “API” refers to active pharmaceutical ingredient. The API used in the tablets according to the invention is 6-((5-methyl-3-(6-methylpyridin-3-yl)isoxazol-4-yl)methoxy)-N-(tetrahydropyran-4-yl)pyridazine-3-carboxamide (I), or a pharmaceutically acceptable salt thereof, also known as alogabat or R07017773.

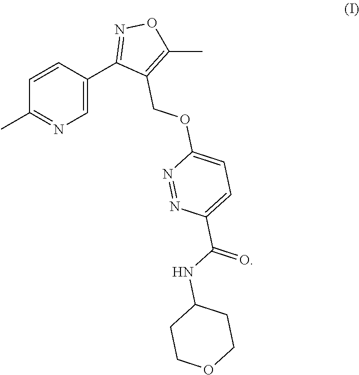 [SMILES: Cc1ccc(-c2noc(C)c2COc2ccc(C(=O)NC3CCOCC3)nn2)cn1]

As used herein, the term “acidic” means having a pH<7.

As used herein, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein, the term “preventing” includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

A s used herein, the term “patient” refers to a human.

### Tablet Formulations

In a first aspect, the present invention provides a tablet comprising a kernel and optionally a coating, wherein said kernel comprises:
(i) the API 6-((5-methyl-3-(6-methylpyridin-3-yl)isoxazol-4-yl)methoxy)-N-(tetrahydropyran-4-yl)pyridazine-3-carboxamide (I), or a pharmaceutically acceptable salt thereof,

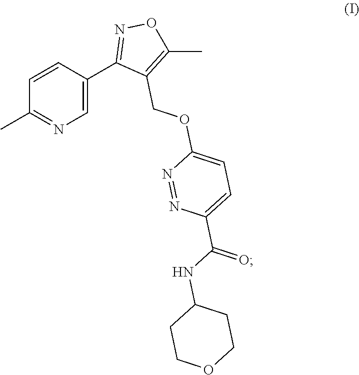 [SMILES: Cc1ccc(-c2noc(C)c2COc2ccc(C(=O)NC3CCOCC3)nn2)cn1]

(ii) a superdisintegrant;
(iii) a first filler;
(iv) a second filler;
(v) a glidant; and
(vi) a lubricant;
wherein the amount of API is ≤25%±1% relative to the total weight of the kernel, preferably ≤20%±1% relative to the total weight of the kernel; and
wherein said kernel does not comprise lactose.

In one embodiment, the invention provides a tablet as described herein, wherein
(i) the weight of said API represents 2%±1% to 20%±1% of the total weight of the kernel;
(ii) the weight of said superdisintegrant represents 3%±1% of the total weight of the kernel;
(iii) the weight of said first filler represents 46%±1% to 57%±1% of the total weight of the kernel;
(iv) the weight of said second filler represents 26%±1% to 32%±1% of the total weight of the kernel;
(v) the weight of said glidant represents 2%±1% of the total weight of the kernel; and
(vi) the weight of said lubricant represents 3%±1% of the total weight of the kernel.

In one embodiment, the invention provides a tablet as described herein, wherein
(i) the weight of said API represents 20%±1% of the total weight of the kernel;
(ii) the weight of said superdisintegrant represents 3%±1% of the total weight of the kernel;
(iii) the weight of said first filler represents 46%±1% of the total weight of the kernel;
(iv) the weight of said second filler represents 26%±1% of the total weight of the kernel;
(v) the weight of said glidant represents 2%±1% of the total weight of the kernel; and
(vi) the weight of said lubricant represents 3%±1% of the total weight of the kernel.

As part of the Phase I clinical studies, a taste assessment with five healthy volunteers was conducted, which revealed that the API has an unpleasant bitter taste. This is an additional problem that is addressed by the tablets of the present invention. Thus, it was found that the API has a low solubility at acidic pH. Consequently, when a tablet according to the invention is dispersed in an acidic beverage, such as apple juice, the API will not dissolve, preventing it from unfolding its unpleasant taste.

In one aspect, the present invention provides a method for administering a tablet according to the invention to a patient, comprising dispersing the tablet in an acidic beverage.

In a preferred embodiment, said acidic beverage is apple juice.

As an alternative to dispersing the tablet in an acidic beverage, the bitter taste of the API may be masked by adding a sweetener and/or a flavor to the tablet formulation. This is particularly useful in case the patient prefers to swallow the tablet as a whole, rather than dispersing it in a liquid prior to administration.

Thus, in one embodiment, the present invention provides a tablet as described herein, further comprising:
(vii) a sweetener.

In one embodiment, the weight of said sweetener represents 0.3%±0.1% to 0.5%±0.1% of the total weight of the kernel.

In one embodiment, said sweetener is sucralose.

In one embodiment, the present invention provides a tablet as described herein, wherein
(i) the weight of said API represents 2%±1% of the total weight of the kernel;
(ii) the weight of said superdisintegrant represents 3%±1% of the total weight of the kernel;
(iii) the weight of said first filler represents 57%±1% of the total weight of the kernel;
(iv) the weight of said second filler represents 32%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

In one embodiment, the present invention provides a tablet as described herein, wherein
(i) the weight of said API represents 3%±1% of the total weight of the kernel;
(ii) the weight of said superdisintegrant represents 3%±1% of the total weight of the kernel;
(iii) the weight of said first filler represents 57%±1% of the total weight of the kernel;
(iv) the weight of said second filler represents 32%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

In one embodiment, the present invention provides a tablet as described herein, wherein
(i) the weight of said API represents 8%±1% of the total weight of the kernel;
(ii) the weight of said superdisintegrant represents 3%±1% of the total weight of the kernel;
(iii) the weight of said first filler represents 54%±1% of the total weight of the kernel;
(iv) the weight of said second filler represents 30%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

In one embodiment, the present invention provides a tablet as described herein, wherein
(i) the weight of said API represents 16%±1% of the total weight of the kernel;
(ii) the weight of said superdisintegrant represents 3%±1% of the total weight of the kernel;
(iii) the weight of said first filler represents 48%±1% of the total weight of the kernel;
(iv) the weight of said second filler represents 27%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

In one embodiment, the present invention provides a tablet as described herein, further comprising:
(viii) a flavor.

In one embodiment, the weight of said flavor represents 1%±0.5% of the total weight of the kernel.

In one embodiment, said flavor is strawberry flavor.

In one embodiment, the present invention provides a tablet as described herein, further comprising:
(vii) a sweetener; and
(viii) a flavor.

In one embodiment, the present invention provides a tablet as described herein, wherein:
(i) the weight of said API represents 20%±1% of the total weight of the kernel;
(ii) the weight of said superdisintegrant represents 3%±1% of the total weight of the kernel;
(iii) the weight of said first filler represents 45%±1% of the total weight of the kernel;
(iv) the weight of said second filler represents 26%±1% of the total weight of the kernel;
(v) the weight of said glidant represents 2%±1% of the total weight of the kernel;
(vi) the weight of said lubricant represents 3%±1% of the total weight of the kernel;
(vii) the weight of said sweetener represents 0.5%±0.1% of the total weight of the kernel; and
(viii) the weight of said flavor represents 1%±0.5% of the total weight of the kernel.

In one embodiment, the present invention provides a tablet as described herein, wherein:
(ii) said disintegrant is croscarmellose sodium;
(iii) said first filler is isomalt;
(iv) said second filler is microcrystalline cellulose;
(v) said glidant is colloidal silicon dioxide; and
(vi) said lubricant is sodium stearyl fumarate.

In one embodiment, the present invention provides a tablet as described herein, wherein the dose strength is 0.5 mg to 375 mg, preferably 2.5 mg to 375 mg, more preferably 3 mg to 200 mg, more preferably 3 mg to 150 mg, more preferably 3 mg to 125 mg, for example 3 mg, 4 mg, 5 mg, 10 mg, 20 mg, 25 mg, or 125 mg.

In one embodiment, the present invention provides a tablet as described herein, wherein the dose strength is 2.5 mg to 375 mg.

In a preferred embodiment, the present invention provides a tablet as described herein, wherein the dose strength is 3 mg to 125 mg.

In a particularly preferred embodiment, the present invention provides a tablet as described herein, wherein the dose strength is 3 mg.

In a particularly preferred embodiment, the present invention provides a tablet as described herein, wherein the dose strength is 4 mg.

In a particularly preferred embodiment, the present invention provides a tablet as described herein, wherein the dose strength is 5 mg.

In a particularly preferred embodiment, the present invention provides a tablet as described herein, wherein the dose strength is 10 mg.

In a particularly preferred embodiment, the present invention provides a tablet as described herein, wherein the dose strength is 20 mg.

In a particularly preferred embodiment, the present invention provides a tablet as described herein, wherein the dose strength is 25 mg.

In a particularly preferred embodiment, the present invention provides a tablet as described herein, wherein the dose strength is 125 mg.

In a preferred embodiment, the present invention provides a tablet as described herein, comprising a coating.

In one embodiment, the weight of said coating is 3%±1% of the weight of the kernel.

In one embodiment, said coating comprises:
(i) hydroxypropyl methylcellulose;
(ii) lactose monohydrate;
(iii) titanium dioxide; and
(iv) polyethylene glylcol 3350.

In one embodiment, in the coating:
(i) the weight of said hydroxypropyl methylcellulose represents 34%±1% of the total weight of the coating;
(ii) the weight of said lactose monohydrate represents 28%±1% of the total weight of the coating;
(iii) the weight of said titanium dioxide represents 26%±1% of the total weight of the coating; and
(iv) the weight of said polyethylene glylcol 3350 represents 12%±1% of the total weight of the coating.

In a preferred embodiment, said coating is Opadry II white 32F280008.

In a particularly preferred embodiment, the present invention provides a tablet which is:

| Component | Amount (mg/tablet) | Function | Quality Standard |
|---|---|---|---|
| Tablet Core | | | |
| API | 125 | API | in-house |
| Isomalt | 288 | Filler | USP/NF, Ph. Eur. |
| Microcrystalline Cellulose | 162 | Filler | USP/NF, Ph. Eur. |
| Croscarmellose Sodium non | 18.75 | Superdisintegrant | USP/NF, Ph. Eur. |
| GMO | | | |
| Colloidal Silicon Dioxide | 12.50 | Glidant | USP/NF, Ph. Eur. |
| Sodium Stearyl Fumarate | 18.75 | Lubricant | USP/NF, Ph. Eur. |
| Subtotal Weight (tablet core) | 625 | - | - |
| Film Coat ^{a} | | Coating agent | |
| Hydroxypropyl Methylcellulose | 6.375 | | USP/NF, Ph. Eur. |
| Lactose Monohydrate | 5.25 | | USP/NF, Ph. Eur. |
| Titanium Dioxide | 4.875 | | USP/NF, Ph. Eur. |
| Polyethylene Glycol 3350 | 2.25 | | USP/NF, Ph. Eur. |
| Purified Water^{b} | | | USP/NF, Ph. Eur. |
| Subtotal Weight (film coat) | 18.75 | - | - |
| Total Weight | 643.75 | - | - |

^{a} A commercially available film-coating mixture (e.g., Opadry [32F280008]) may be used;
it has thesame composition as that described above.
^{b} Purified water (USP/NF, Ph.Eur.) is used for aqueous film coating;
it is essentially removed during processing.

In a particularly preferred embodiment, the present invention provides a tablet which is:

| Component | Amount (mg/tablet) | Function | Quality Standard |
|---|---|---|---|
| Tablet Core | | | |
| API | 125 | API | in-house |
| Isomalt | 281 | Filler | USP/NF, Ph. Eur. |
| Microcrystalline Cellulose | 158.065 | Filler | USP/NF, Ph. Eur. |
| Croscarmellose Sodium non | 18.75 | Superdisintegrant | USP/NF, Ph. Eur. |
| GMO | | | |
| Colloidal Silicon Dioxide | 12.50 | Glidant | USP/NF, Ph. Eur. |
| Sodium Stearyl Fumarate | 18.75 | Lubricant | USP/NF, Ph. Eur. |
| Sucralose | 3.125 | Sweetener | USP/NF, Ph. Eur. |
| Strawberry Flavor | 7.81 | Flavor | In-house |
| Subtotal Weight (tablet core) | 625 | - | - |
| Film Coat ^{a} | | Coating agent | |
| Hydroxypropyl Methylcellulose | 6.375 | | USP/NF, Ph. Eur. |
| Lactose Monohydrate | 5.25 | | USP/NF, Ph. Eur. |
| Titanium Dioxide | 4.875 | | USP/NF, Ph. Eur. |
| Polyethylene Glycol 3350 | 2.25 | | USP/NF, Ph. Eur. |
| Purified Water^{b} | | | USP/NF, Ph. Eur. |
| Subtotal Weight (film coat) | 18.75 | - | - |
| Total Weight | 643.75 | - | - |

^{a} A commercially available film-coating mixture (e.g., Opadry [32F280008]) may be used;
it has the same composition as that described above.
^{b} Purified water (USP, Ph.Eur.) is used for aqueous film coating;
it is essentially removed during processing.

In a particularly preferred embodiment, the present invention provides a tablet which is:

| Component | Amount (mg/tablet) | Function | Quality Standard |
|---|---|---|---|
| Tablet Core | | | |
| API | 20.00 | API | in-house |
| Isomalt | 60.60 | Filler | USP/NF, Ph. Eur. |
| Microcrystalline Cellulose | 34.09 | Filler | USP/NF, Ph. Eur. |
| Croscarmellose Sodium non | 3.75 | Superdisintegrant | USP/NF, Ph. Eur. |
| GMO | | | |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | Coating agent | |
| Hydroxypropyl Methylcellulose | 1.275 | | USP/NF, Ph. Eur. |
| Lactose Monohydrate | 1.05 | | USP/NF, Ph. Eur. |
| Titanium Dioxide | 0.975 | | USP/NF, Ph. Eur. |
| Polyethylene Glycol 3350 | 0.45 | | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} A commercially available film-coating mixture (e.g., Opadry [32F280008]) may be used;
it has the same composition as that described above.
^{b} Purified water (USP/NF, Ph.Eur.) is used for aqueous film coating;
it is essentially removed during processing.

In a particularly preferred embodiment, the present invention provides a tablet which is:

| Component | Amount (mg/tablet) | Function | Quality Standard |
|---|---|---|---|
| Tablet Core | | | |
| API | 20.00 | API | in-house |
| Isomalt | 60.60 | Filler | USP/NF, Ph. Eur. |
| Microcrystalline Cellulose | 34.09 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

In a particularly preferred embodiment, the present invention provides a tablet which is:

| Component | Amount (mg/tablet) | Function | Quality Standard |
|---|---|---|---|
| Tablet Core | | | |
| API | 10.00 | API | in-house |
| Isomalt | 67.00 | Filler | USP/NF, Ph. Eur. |
| Microcrystalline Cellulose | 37.69 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Waterª | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

In a particularly preferred embodiment, the present invention provides a tablet which is:

| Component | Amount (mg/tablet) | Function | Quality Standard |
|---|---|---|---|
| Tablet Core | | | |
| API | 4.00 | API | in-house |
| Isomalt | 70.84 | Filler | USP/NF, Ph. Eur. |
| Microcrystalline Cellulose | 39.84 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

In a particularly preferred embodiment, the present invention provides a tablet which is:

| Component | Amount (mg/tablet) | Function | Quality Standard |
|---|---|---|---|
| Tablet Core | | | |
| API | 3.00 | API | in-house |
| Isomalt | 71.5 | Filler | USP/NF, Ph. Eur. |
| Microcrystalline Cellulose | 40.21 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

As outlined above, it was found to be important that the tablet according to the invention be dispersible in water and acidic beverages, such as apple juice. This requirement was achieved, i.a., by adding a superdisintegrant to the formulation. In a particularly preferred embodiment, the present invention provides a tablet as described herein, wherein the tablet has a disintegration time of <3 minutes in purified water at 37° C. (see Example 3).

### Manufacturing Process

In one aspect, the present invention provides a process for manufacturing a tablet described herein, comprising:
(i) Weighing the API and the first filler into a container and screening the two components to afford a blend;
(ii) Adding the second filler, the superdisintegrant, the glidant, and optionally a sweetener and/or a flavor to the blend from step (i) and screening all components;
(iii) Blending the screened material from step (ii) to afford a powder blend;
(iv) Screening the lubricant, adding it to the powder blend from step (iii) and blending;
(v) Screening the material from step (iv);
(vi) Blending the screened material from step (v);
(vii) Compressing the blend from step (vi) into tablet kernels; and
(viii) Spraying a film coating suspension onto the tablet kernels from step (vii).

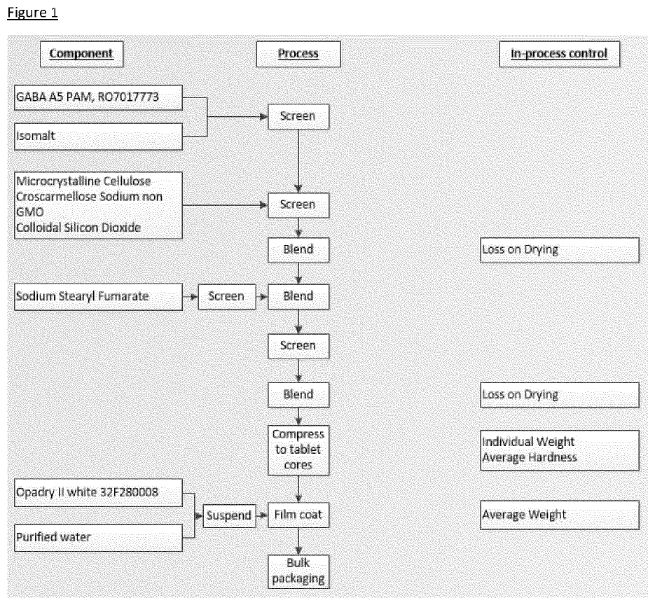

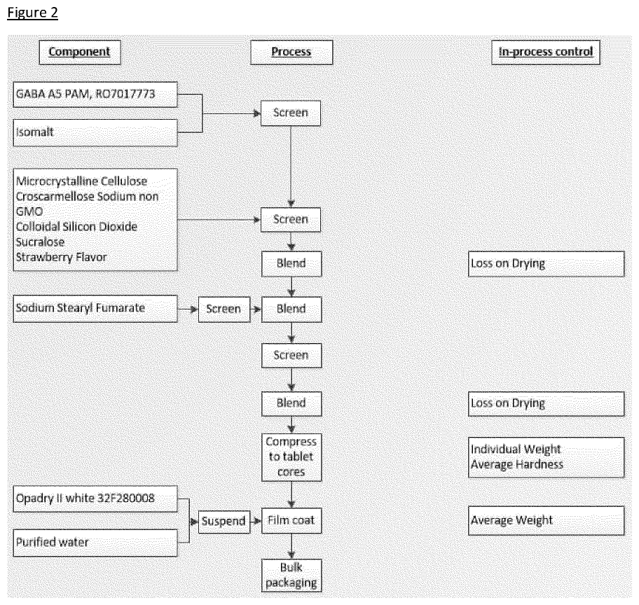

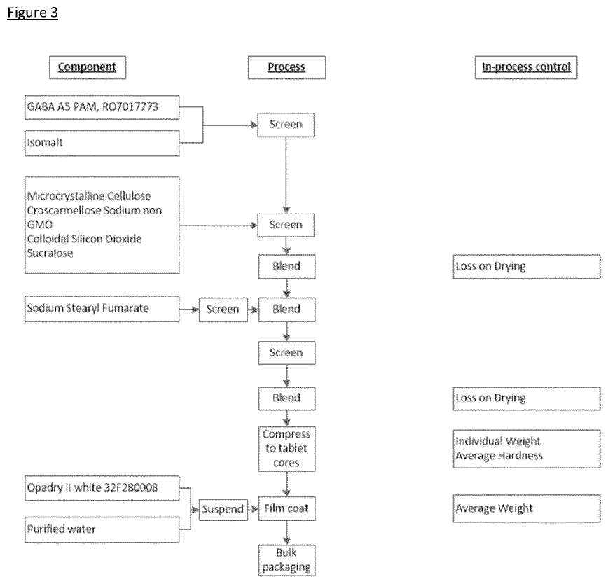

In a preferred embodiment, the process according to the invention is as shown in FIGS. 1-3.

### Uses

In one aspect, the present invention provides a tablet as described herein, for use as a medicament.

In one aspect, the present invention provides a method for treating or preventing a GABA_{A} α5 receptor related disease in a patient, comprising administering one or more tablets described herein to the patient.

In one aspect, the present invention provides a tablet described herein, for use in a method of treating or preventing a GABA_{A} α5 receptor related disease in a patient.

In one aspect, the present invention provides the use of a tablet described herein in a method of treating or preventing a GABA_{A} α5 receptor related disease in a patient.

In one embodiment, said GABA_{A} α5 receptor related disease is selected from Alzheimer's disease, mild cognitive impairment (MCI), age-related cognitive decline, negative and/or cognitive symptoms associated with schizophrenia, bipolar disorders, autism spectrum disorder (ASD), Angelman syndrome, Rett syndrome, Prader-Willi syndrome, epilepsy, post-traumatic stress disorder (PTSD), amyotrophic lateral sclerosis (ALS), and fragile-X disorder.

In a preferred embodiment, said GABA_{A} α5 receptor related disease is selected from autism spectrum disorder and Angelman syndrome.

In a particularly preferred embodiment, said GABA_{A} α5 receptor related disease is autism spectrum disorder.

In a particularly preferred embodiment, said GABA_{A} 5 receptor related disease is Angelman syndrome.

### EXAMPLES

The following examples are provided for illustration of the invention. They should not be considered as limiting the scope of the invention, but merely as being representative thereof.

### Example 1—Tablet Dissolution Behaviour

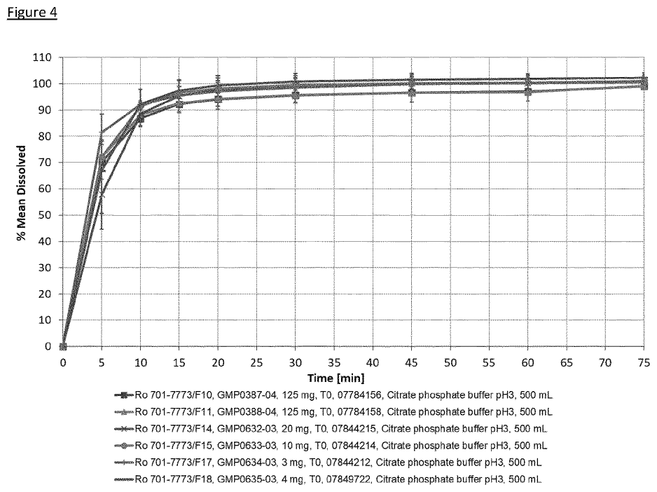

FIG. 4 highlights the release behavior of the formulations according to the invention in 500 mL citrate buffer. The dissolution was determined according to USP 711 and harmonized with Ph. Eur. 2.9.3, using a paddle apparatus. The test preparations were then tested by HPLC.

### Example 2—Manufacturability (Tablet Hardness/Solid Fraction Profiles)

The manufacturability of the tablets was assessed on a representative batch by using the highest dose strength of 20 mg (see Example 7) since this is presumed most critical for the formulation performance. The solid fraction (SF) is the fraction of the volume of the compact that is solid. The porosity is one minus the solid fraction. A change of 10% to 20% in a mechanical property may occur with each 0.01 change in solid fraction. Therefore, it is critical to compare materials at the same solid fraction (Hiestand, E. N. and Smith, D. P., (1984). “Indices of tableting performance”, Powder Technology, 38(2), pp. 145-159).

TABLE t

| hardness | Solid Fraction |
|---|---|
| 18 N | 0.70 |
| 34 N | 0.75 |
| 46 N | 0.78 |
| 55 N | 0.81 |

### Example 3—Tablet Disintegration Behaviour in Dependence of Tablet Hardness

The tablet disintegration behaviour was monitored during routine manufacturing of the batches. The disintegration was performed using “Apparatus A” method in purified water at 37° C., adapted from USP 701 and Ph. Eur. 2.9.1 for dispersible tablets.

| | Tablet | Disintegration |
|---|---|---|
| Formulation | hardness | time at 37° C. |
| 20 mg Film-coated | 72 N | 2:20 min: sek |
| tablet | | |
| 10 mg Film-coated | 73 N | 2:30 min: sek |
| tablet | | |
| 4 mg Film-coated | 74 N | 2:34 min: sek |
| tablet | | |
| 3 mg Film-coated | 61 N | 1:54 min:sek |
| tablet | | |

### Example 4—Plastic Behaviour of API

The measurements were performed on cubic samples compressed at 3 different solid fractions in triplicate (duplicate or single coupons for limited amounts). The dynamic response of the materials was characterized by a high-speed indentation with a spherical pendulum that impacts on a mayor face of the specimen (dynamic hardness Hd).

In summary, it is important to highlight the extreme plastic behaviour of the API even at a lower compaction pressure, which is represented by the dynamic hardness (Hd) of 40.4 MPa, which is out of the desirable range of 100-200 MPa (Amidon, G. E., (1995), “Physical and mechanical property characterization of powders”, In Brittain HG, editor Physical Characterization of Pharmaceutical).

The tensile strength of a compact was determined by measuring the force necessary to cause tensile failure under transverse compression. This is analogous to the measurement of tablet crushing force or hardness. The experimental conditions and compact formation are well controlled in these studies, making results more reproducible and quantitative. Desirable tensile strengths that indicate strong compact formation are >1 MPa. It was fund that the present API has a low tensile strength value of about 0.79 MPa.

In summary, due to the plastic behaviour and low tensile strength value of the API, the API is not compatible with excipients that exhibit a low tensile strength- and a high compaction pressure value, such as lactose, especially in formulations with a high drug load.

### Example 5—125 mg Film-Coated Tablet

| Component | Amount (mg/tablet) | Function | Quality Standard |
|---|---|---|---|
| Tablet Core | | | |
| API | 125 | API | in-house |
| Isomalt | 288 | Filler | USP/NF, Ph. Eur. |
| Microcrystalline Cellulose | 162 | Filler | USP/NF, Ph. Eur. |
| Croscarmellose Sodium non | 18.75 | Superdisintegrant | USP/NF, Ph. Eur. |
| GMO | | | |
| Colloidal Silicon Dioxide | 12.50 | Glidant | USP/NF, Ph. Eur. |
| Sodium Stearyl Fumarate | 18.75 | Lubricant | USP/NF, Ph. Eur. |
| Subtotal Weight (tablet core) | 625 | - | - |
| Film Coat a | | Coating agent | |
| Hydroxypropyl Methylcellulose | 6.375 | | USP/NF, Ph. Eur. |
| Lactose Monohydrate | 5.25 | | USP/NF, Ph. Eur. |
| Titanium Dioxide | 4.875 | | USP/NF, Ph. Eur. |
| Polyethylene Glycol 3350 | 2.25 | | USP/NF, Ph. Eur. |
| Purified Waterb | | | USP/NF, Ph. Eur. |
| Subtotal Weight (film coat) | 18.75 | - | - |
| Total Weight | 643.75 | - | - |

^{a} A commercially available film-coating mixture (e.g., Opadry [32F280008]) may be used;
it has the same composition as that described above.
^{b} Purified water (USP/NF, Ph.Eur.) is used for aqueous film coating;
it is essentially removed during processing.

### Example 6—Sweetened and Flavoured 125 mg Film-Coated Tablet

| Component | Amount (mg/tablet) | Function | Quality Standard |
|---|---|---|---|
| Tablet Core | | | |
| API | 125 | API | in-house |
| Isomalt | 281 | Filler | USP/NF, Ph. Eur. |
| Microcrystalline Cellulose | 158.065 | Filler | USP/NF, Ph. Eur. |
| Croscarmellose Sodium non | 18.75 | Superdisintegrant | USP/NF, Ph. Eur. |
| GMO | | | |
| Colloidal Silicon Dioxide | 12.50 | Glidant | USP/NF, Ph. Eur. |
| Sodium Stearyl Fumarate | 18.75 | Lubricant | USP/NF, Ph. Eur. |
| Sucralose | 3.125 | Sweetener | USP/NF, Ph. Eur. |
| Strawberry Flavor | 7.81 | Flavor | In-house |
| Subtotal Weight (tablet core) | 625 | | - |
| Film Coat a | | Coating agent | |
| Hydroxypropyl Methylcellulose | 6.375 | | USP/NF, Ph. Eur. |
| Lactose Monohydrate | 5.25 | | USP/NF, Ph. Eur. |
| Titanium Dioxide | 4.875 | | USP/NF, Ph. Eur. |
| Polyethylene Glycol 3350 | 2.25 | | USP/NF, Ph. Eur. |
| Purified Waterb | | | USP/NF, Ph. Eur. |
| Subtotal Weight (film coat) | 18.75 | - | - |
| Total Weight | 643.75 | - | - |

^{a} A commercially available film-coating mixture (e.g., Opadry [32F280008]) may be used;
it has the same composition as that described above.
^{b} Purified water (USP, Ph.Eur.) is used for aqueous film coating;
it is essentially removed during processing.

### Example 7—Sweetened 20 mg Film-Coated Tablet

| Component | Amount (mg/tablet) | Function | Quality Standard |
|---|---|---|---|
| Tablet Core | | | |
| API | 20.00 | API | in-house |
| Isomalt | 60.60 | Filler | USP/NF, Ph. Eur. |
| Microcrystalline Cellulose | 34.09 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

### Example 8—Sweetened 10 mg Film-Coated Tablet

| Component | Amount (mg/tablet) | Function | Quality Standard |
|---|---|---|---|
| Tablet Core | | | |
| API | 10.00 | API | in-house |
| Isomalt | 67.00 | Filler | USP/NF, Ph. Eur. |
| Microcrystalline Cellulose | 37.69 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

### Example 9—Sweetened 4 mg Film-Coated Tablet

| Component | Amount (mg/tablet) | Function | Quality Standard |
|---|---|---|---|
| Tablet Core | | | |
| API | 4.00 | API | in-house |
| Isomalt | 70.84 | Filler | USP/NF, Ph. Eur. |
| Microcrystalline Cellulose | 39.84 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

### Example 10—Sweetened 3 mg Film-Coated Tablet

| Component | Amount (mg/tablet) | Function | Quality Standard |
|---|---|---|---|
| Tablet Core | | | |
| API | 3.00 | API | in-house |
| Isomalt | 71.5 | Filler | USP/NF, Ph. Eur. |
| Microcrystalline Cellulose | 40.21 | Filler | USP/NF, Ph. Eur. |
| Croscarmellose Sodium non | 3.75 | Superdisintegrant | USP/NF, Ph. Eur. |
| GMO | | | |
| Sucralose | 0.31 | Sweetener | USP/NF, Ph. Eur. |
| Colloidal Silicon Dioxide | 2.50 | Glidant | USP/NF, Ph. Eur. |
| Sodium Stearyl Fumarate | 3.75 | Lubricant | USP/NF, Ph. Eur. |
| Subtotal Weight (tablet core) | 125.00 | - | - |
| Film Coat | | | |
| Opadry II white 32F280008 | 3.75 | Coating agent | USP/NF, Ph. Eur. |
| Purified Watera | | | USP/NF, Ph. Eur. |
| Subtotal Weight (film coat) | 3.75 | - | - |
| Total Weight | 128.75 | - | - |

^{a} Purified water (USP/NF, Ph.Eur.) is used for aqueous film coating;
it is essentially removed during processing.

### Example 11—Process for Tablet Manufacturing

Processes for manufacturing the tablets according to the invention are shown in FIGS. 1-3.

The process steps can be summarized as follows:
(i) Weighing the API and isomalt into a container and screening the two components to afford a blend;
(ii) Adding microcrystalline cellulose, croscarmellose sodium, colloidal silicon dioxide, and optionally sucralose and/or strawberry flavor to the blend from step (i) and screening all components;
(iii) Blending the screened material from step (ii) to afford a powder blend;
(iv) Screening sodium stearyl fumarate, adding it to the powder blend from step (iii) and blending;
(v) Screening the material from step (iv);
(vi) Blending the screened material from step (v);
(vii) Compressing the blend from step (vi) into tablet kernels; and
(viii) Spraying a film coating suspension onto the tablet kernels from step (vii).
   Screening can be performed mechanically or manually, as needed.
   Preferably, the blend from step (vi) is compressed into tablet kernels by direct compression.

### Equipment

The container used in step (i) was a metal drum. Powder blends were mixed with a Servolift blender. A Korsch XL100 Wipcon rotary tableting press was used to press tablet kernels by direct compression. Film coating was performed using a Glatt GMPC 1 apparatus.

1. A tablet comprising a kernel and optionally a coating, wherein said kernel comprises:
(i) the API 6-((5-methyl-3-(6-methylpyridin-3-yl)isoxazol-4-yl)methoxy)-N-(tetrahydropyran-4-yl)pyridazine-3-carboxamide (I), or a pharmaceutically acceptable salt thereof, 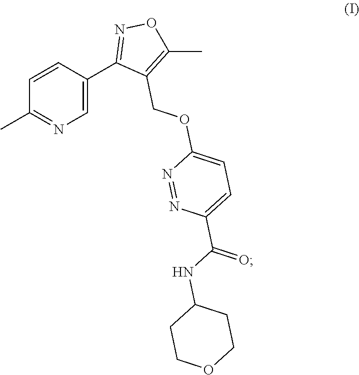 [SMILES: Cc1ccc(-c2noc(C)c2COc2ccc(C(=O)NC3CCOCC3)nn2)cn1]
(ii) a superdisintegrant;
(iii) a first filler;
(iv) a second filler;
(v) a glidant; and
(vi) a lubricant;
wherein the amount of API is ≤25%±1% relative to the total weight of the kernel, preferably ≤20%±1% relative to the total weight of the kernel; and
wherein said kernel does not comprise lactose.

2. The tablet according to claim 1, wherein
(i) the weight of said API represents 2%±1% to 20%±1% of the total weight of the kernel;
(ii) the weight of said superdisintegrant represents 3%±1% of the total weight of the kernel;
(iii) the weight of said first filler represents 46%±1% to 57%±1% of the total weight of the kernel;
(iv) the weight of said second filler represents 26%±1% to 32%±1% of the total weight of the kernel;
(v) the weight of said glidant represents 2%±1% of the total weight of the kernel; and
(vi) the weight of said lubricant represents 3%±1% of the total weight of the kernel.

3. The tablet according to claim 1, wherein
(i) the weight of said API represents 20%±1% of the total weight of the kernel;
(ii) the weight of said superdisintegrant represents 3%±1% of the total weight of the kernel;
(iii) the weight of said first filler represents 46%±1% of the total weight of the kernel;
(iv) the weight of said second filler represents 26%±1% of the total weight of the kernel;
(v) the weight of said glidant represents 2%±1% of the total weight of the kernel; and
(vi) the weight of said lubricant represents 3%±1% of the total weight of the kernel.

4. The tablet according to claim 1 or 2, further comprising:
(vii) a sweetener.

5. The tablet according to claim 4, wherein the weight of said sweetener represents 0.3% 0.1% to 0.5%±0.1% of the total weight of the kernel.

6. The tablet according to claim 4, wherein
(i) the weight of said API represents 2%±1% of the total weight of the kernel;
(ii) the weight of said superdisintegrant represents 3%±1% of the total weight of the kernel;
(iii) the weight of said first filler represents 57%±1% of the total weight of the kernel;
(iv) the weight of said second filler represents 32%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

7. The tablet according to claim 4, wherein
(i) the weight of said API represents 3%±1% of the total weight of the kernel;
(ii) the weight of said superdisintegrant represents 3%±1% of the total weight of the kernel;
(iii) the weight of said first filler represents 57%±1% of the total weight of the kernel;
(iv) the weight of said second filler represents 32%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

8. The tablet according to claim 4, wherein
(i) the weight of said API represents 8%±1% of the total weight of the kernel;
(ii) the weight of said superdisintegrant represents 3%±1% of the total weight of the kernel;
(iii) the weight of said first filler represents 54%±1% of the total weight of the kernel;
(iv) the weight of said second filler represents 30%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

9. The tablet according to claim 4, wherein
(i) the weight of said API represents 16%±1% of the total weight of the kernel;
(ii) the weight of said superdisintegrant represents 3%±1% of the total weight of the kernel;
(iii) the weight of said first filler represents 48%±1% of the total weight of the kernel;
(iv) the weight of said second filler represents 27%±1% of the total weight of the kernel;
(v) the weight of said glidant represents 2%±1% of the total weight of the kernel;
(vi) the weight of said lubricant represents 3%±1% of the total weight of the kernel; and
(vii) the weight of said sweetener represents 0.3%±0.1% of the total weight of the kernel.

10. The tablet according to any one of claims 1, 2, 4 and 5, further comprising:
(viii) a flavor.

11. The tablet according to claim 10, wherein the weight of said flavor represents 1%±0.5% of the total weight of the kernel.

12. The tablet according to claim 10, wherein
(i) the weight of said API represents 20%±1% of the total weight of the kernel;
(ii) the weight of said superdisintegrant represents 3%±1% of the total weight of the kernel;
(iii) the weight of said first filler represents 45%±1% of the total weight of the kernel;
(iv) the weight of said second filler represents 26%±1% of the total weight of the kernel;
(v) the weight of said glidant represents 2%±1% of the total weight of the kernel;
(vi) the weight of said lubricant represents 3%±1% of the total weight of the kernel;
(vii) the weight of said sweetener represents 0.5%±0.1% of the total weight of the kernel; and
(viii) the weight of said flavor represents 1%±0.5% of the total weight of the kernel.

13. The e tablet according to any one of claims 1 to 12, wherein:
(ii) said superdisintegrant is croscarmellose sodium;
(iii) said first filler is isomalt;
(iv) said second filler is microcrystalline cellulose;
(v) said glidant is colloidal silicon dioxide; and
(vi) said lubricant is sodium stearyl fumarate.

14. The tablet according to any one of claims 4 to 12, wherein said sweetener is sucralose.

15. The tablet according to any one of claims 10 to 12, wherein said flavor is strawberry flavor.

16. The tablet according to any one of claims 1 to 15, wherein the dose strength is 0.5 mg to 375 mg, preferably 2.5 mg to 375 mg, more preferably 3 mg to 200 mg, more preferably 3 mg to 150 mg, more preferably 3 mg to 125 mg, for example 3 mg, 4 mg, 5 mg, 10 mg, 20 mg, 25 mg, or 125 mg.

17. The tablet according to any one of claims 1 to 16, comprising a coating.

18. The tablet according to claim 17, wherein the weight of said coating is 3%±1% of the weight of the kernel.

19. The tablet according to claim 17 or 18, wherein said coating comprises:
(i) hydroxypropyl methylcellulose;
(ii) lactose monohydrate;
(iii) titanium dioxide; and
(iv) polyethylene glylcol 3350.

20. The tablet according to claim 19, wherein:
(i) the weight of said hydroxypropyl methylcellulose represents 34%±1% of the total weight of the coating;
(ii) the weight of said lactose monohydrate represents 28%±1% of the total weight of the coating;
(iii) the weight of said titanium dioxide represents 26%±1% of the total weight of the coating; and
(iv) the weight of said polyethylene glylcol 3350 represents 12%±1% of the total weight of the coating.

21. The tablet according to any one of claims 1 to 20, wherein the tablet has a disintegration time of <3 minutes in purified water at 37° C.

22. A process for manufacturing a tablet according to any one of claims 1 to 21, comprising:
(i) Weighing the API and the first filler into a container and screening the two components to afford a blend;
(ii) Adding the second filler, the superdisintegrant, the glidant, and optionally a sweetener and/or a flavor to the blend from step (i) and screening all components;
(iii) Blending the screened material from step (ii) to afford a powder blend;
(iv) Screening the lubricant, adding it to the powder blend from step (iii) and blending;
(v) Screening the material from step (iv);
(vi) Blending the screened material from step (v);
(vii) Compressing the blend from step (vi) into tablet kernels; and
(viii) Spraying a film coating suspension onto the tablet kernels from step (vii).

23. A kit comprising a tablet according to any one of claims 1 to 21 and instructions for using the tablet.

24. A tablet according to any one of claims 1 to 21, for use as a medicament.

25. A method for treating or preventing a GABA_{A} α5 receptor related disease in a patient, comprising administering one or more tablets according to any one of claims 1 to 21 to the patient.

26. The method according to claim 25, wherein said administration comprises dispersing the one or more tablet in a liquid.

27. The method according to claim 26, wherein said liquid is water or an acidic beverage, in particular apple juice.

28. A tablet according to any one of claims 1 to 21, for use in a method according to any one of claims 25 to 27.

29. Use of a tablet according to any one of claims 1 to 21 in a method according to any one of claims 25 to 27.

30. A method for administering a tablet according to any one of claims 1 to 21 to a patient, comprising dispersing the tablet in an acidic beverage.

31. The method according to claim 30, wherein said acidic beverage is apple juice.

32. The invention as described hereinbefore.
